(19) United States
(12) Patent Application Publication
(10) Pub. No.: US 2013/0018264 A1
(43) Pub. Date: Jan. 17, 2013

(54) METHOD AND SYSTEM FOR ULTRASOUND IMAGING

(71) Applicants: Olivier Gerard, Horten (NO); Stein Inge Rabben, Horten (NO); Andreas Michael Ziegler, Horten (NO)

(72) Inventors: Olivier Gerard, Horten (NO); Stein Inge Rabben, Horten (NO); Andreas Michael Ziegler, Horten (NO)

(73) Assignee: GENERAL ELECTRIC COMPANY, Schenectady, NY (US)

(21) Appl. No.: 13/184,104

(22) Filed: Jul. 15, 2011

(51) Int. Cl. A61B 8/13

(52) U.S. Cl. 600/443

(57) ABSTRACT

An ultrasound imaging system and method include displaying a first sequence of images generated from first ultrasound data, where the first ultrasound data includes volumetric ultrasound data. The system and method include selecting an acquisition target from the first sequence of images and automatically configuring an acquisition parameter based on the selected acquisition target. The system and method include implementing the acquisition parameter to acquire second ultrasound data of the acquisition target. The system and method include displaying a second sequence of images generated from the second ultrasound data, wherein the second sequence of images is of a higher frame rate than the first sequence of images.

### FIELD OF THE INVENTION

This disclosure relates generally to a method and system for using a first image generated from volumetric ultrasound data to identify an acquisition target in order to then acquire additional ultrasound data of the acquisition target.

### BACKGROUND OF THE INVENTION

Many modem ultrasound imaging systems are capable of acquiring volumetric ultrasound data. Volumetric ultrasound data is typically very useful because it is oftentimes possible to generate an image from volumetric ultrasound data including either all or else a significant portion of an organ. Visualizing an image including an entire organ or a large portion of an organ is useful because it is easier for the user to remain oriented within the image. However, images generated from volumetric ultrasound data still suffer from several limitations. Specifically, images generated from volumetric ultrasound data of a diagnostically useful field-of-view typically suffer from lower spatial resolution and lower temporal resolution than images generated from conventional two-dimensional ultrasound data. Or, conversely, the user may have to accept a much smaller field-of-view in order to increase the spatial resolution and the temporal resolution of the image generated from volumetric ultrasound data. Unfortunately, if a small field-of-view is selected, many of the benefits of using volumetric ultrasound data are negated.

Therefore, for these and other reasons, an improved method of ultrasound imaging and an improved ultrasound imaging system are desired.

### BRIEF DESCRIPTION OF THE INVENTION The above-mentioned shortcomings, disadvantages and problems are addressed herein which will be understood by reading and understanding the following specification.

In an embodiment, a method of ultrasound imaging includes displaying a first sequence of images generated from first ultrasound data, wherein the first ultrasound data includes volumetric ultrasound data. The method includes selecting an acquisition target from the first sequence of images and automatically configuring an acquisition parameter based on the selected acquisition target. The method includes implementing the acquisition parameter to acquire second ultrasound data of the acquisition target. The method includes displaying a second sequence of images generated from the second ultrasound data, wherein the second sequence of images is of a higher frame rate than the first sequence of images.

In another embodiment, a method of ultrasound imaging includes acquiring volumetric ultrasound data, displaying an image generated from the volumetric ultrasound data, and adjusting the position of an icon on the image to control a position of a plane. The method includes automatically configuring an acquisition parameter based on the position of the plane. The method includes implementing the acquisition parameter to acquire two-dimensional ultrasound data at the position of the plane and displaying a two-dimensional image generated from the two-dimensional ultrasound data.

In another embodiment, an ultrasound system includes a probe adapted to scan a volume of interest, a display device, and a processor in electronic communication with the probe and the display device. The processor is configured to control the probe to acquire first ultrasound data, where the first ultrasound data includes volumetric ultrasound data. The processor is configured to display a first image based on the first ultrasound data on the display device. The processor is configured to automatically configure an acquisition parameter based on the selection of an acquisition target in the first image. The processor is configured to implement the acquisition parameter to acquire second ultrasound data of the acquisition target, where the second ultrasound data is of higher temporal resolution than the first ultrasound data. The processor is configured to display an image generated from the second ultrasound data on the display device.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a flow chart illustrating a method in accordance with an embodiment;

FIG. 3 is schematic representation of a first image in accordance with an embodiment;

FIG. 4 is a flow chart illustrating a method in accordance with an embodiment; and

FIG. 5 is a schematic representation of a volume-rendered image and an icon in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

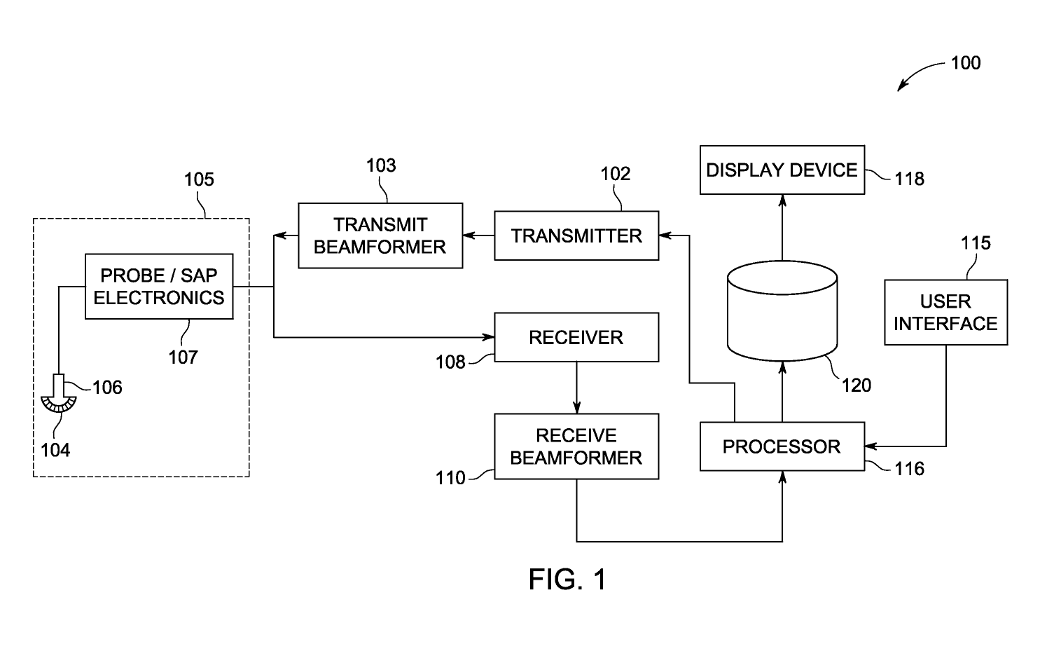

FIG. 1 is a schematic diagram of an ultrasound imaging system 100 in accordance with an embodiment. The ultrasound imaging system 100 includes a transmitter 102 that transmits a signal to a transmit beamformer 103 which in turn drives transducer elements 104 within a transducer array 106 to emit pulsed ultrasonic signals into a structure, such as a patient (not shown). A probe 105 includes the transducer array 106, the transducer elements 104 and probe/SAP electronics 107. The probe/SAP electronics 107 may be used to control the switching of the transducer elements 104. The probe/SAP electronics 107 may also be used to group the elements 104 into one or more sub-apertures. A variety of geometries of transducer arrays may be used. The pulsed ultrasonic signals are back-scattered from structures in the body, like blood cells or muscular tissue, to produce echoes that return to the transducer elements 104. The echoes are converted into electrical signals, or ultrasound data, by the transducer elements 104 and the electrical signals are received by a receiver 108. For purposes of this disclosure, the term ultrasound data may include data that was acquired and/or processed by an ultrasound system. Examples of ultrasound data include volumetric ultrasound data, two-dimensional ultrasound data, and one-dimensional ultrasound data. The electrical signals representing the received echoes are passed through a receive beam-former 110 that outputs ultrasound data. A user interface 115 may be used to control operation of the ultrasound imaging system 100, including, to control the input of patient data, to change a scanning or display parameter, and the like.

The ultrasound imaging system 100 also includes a processor 116 to process the ultrasound data and generate frames or images for display on a display device 118. The processor 116 is in electronic communication with the probe 105 and the display device 118. The processor 116 may be hard-wired to the probe 105 and the display device 118, or the probe may be in electronic communication through other techniques includes wireless communication. The processor 116 may be adapted to perform one or more processing operations according to a plurality of selectable ultrasound modalities on the ultrasound data. Other embodiments may use multiple processors to perform various processing tasks. The processor 116 may also be adapted to control the acquisition of ultrasound data with the probe 105. The ultrasound data may be processed in real-time during a scanning session as the echo signals are received. For purposes of this disclosure, the term “real-time” is defined to include a process performed with no intentional lag or delay. An embodiment may update the displayed ultrasound image at a rate of more than 20 times per second. The images may be displayed as part of a live image. For purposes of this disclosure, the term “live image” is defined to include a dynamic image that updates as additional frames of ultrasound data are acquired. For example, ultrasound data may be acquired even as images are being generated based on previously acquired data and while a live image is being displayed. Then, according to an embodiment, as additional ultrasound data are acquired, additional frames or images generated from more-recently acquired ultrasound data are sequentially displayed. Additionally or alternatively, the ultrasound data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the ultrasound signal while a second processor may be used to further process the data prior to displaying an image. It should be appreciated that other embodiments may use a different arrangement of processors.

Still referring to FIG. 1, the ultrasound imaging system 100 may continuously acquire ultrasound data at a frame rate of, for example, 20 Hz to 150 Hz. However, other embodiments may acquire ultrasound data at a different rate. A memory 120 is included for storing processed frames of acquired ultrasound data that are not scheduled to be displayed immediately. In an exemplary embodiment, the memory 120 is of sufficient capacity to store at least several seconds worth of frames of ultrasound data. The frames of ultrasound data are stored in a manner to facilitate retrieval thereof according to the order or time of acquisition. As described hereinabove, the ultrasound data may be retrieved during the generation and display of a live image. The memory 120 may include any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring ultrasound data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well known by those skilled in the art and will therefore not be described in further detail.

In various embodiments of the present invention, ultrasound information may be processed by other or different mode-related modules. A non-limiting list of modes includes: B-mode, Color Doppler, power Doppler, M-mode, spectral Doppler anatomical M-mode, strain, and strain rate. For example, one or more modules may generate B-mode, color Doppler, power Doppler, M-mode, anatomical M-mode, strain, strain rate, spectral Doppler images and combinations thereof, and the like. The images are stored and timing information indicating a time at which the image was acquired in memory may be recorded with each image. The modules may include, for example, a scan conversion module to perform scan conversion operations to convert the image frames from Polar to Cartesian coordinates. A video processor module may be provided that reads the images from a memory and displays the image in real time while a procedure is being carried out on a patient. A video processor module may store the image in an image memory, from which the images are read and displayed. The ultrasound imaging system 100 shown may include a console system, a cart-based system, or a portable system, such as a hand-held or laptop-style system according to various embodiments.

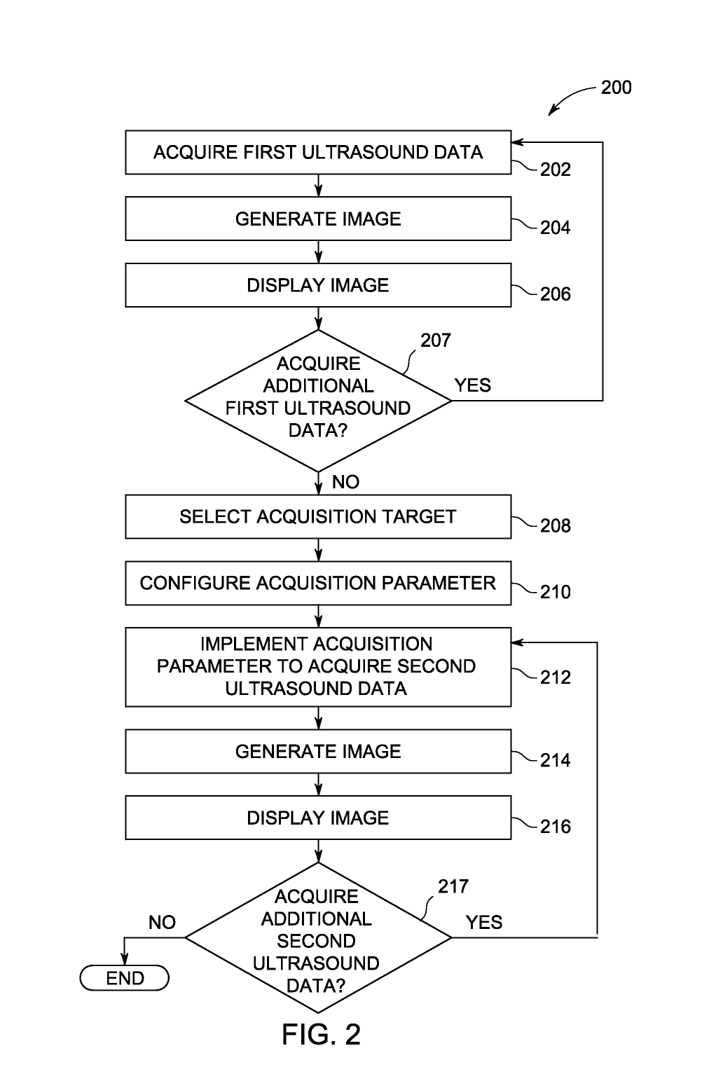

FIG. 2 is a flow chart illustrating a method 200 in accordance with an embodiment. The individual blocks represent steps that may be performed in accordance with the method 200. The technical effect of the method 200 is the display of a second image generated from second ultrasound data in response to the selection of an acquisition target in a first image. The steps of the method 200 will be described according to an exemplary embodiment where the steps are performed with the ultrasound imaging system 100 (shown in FIG. 1).

Referring to both FIG. 1 and FIG. 2, at step 202, the ultrasound imaging system 100 acquires first ultrasound data. The first ultrasound data is volumetric ultrasound data according to an embodiment. For purposes of this disclosure, the term “volumetric ultrasound data” is defined to include ultrasound data of a volume within a patient or subject. Volumetric ultrasound data includes multiple samples in each of three-dimensions. When acquiring volumetric ultrasound data, the processor 116 controls the transmitter 102, the transmit beamformer 103, the probe/sap electronics 107, the receiver 108, and the receive beamformer 110 in order to acquire samples at various positions within a volume. By way of contrast, the samples collected to acquire two-dimensional ultrasound data all lie within generally the same plane. The samples collected to acquire one-dimensional ultrasound data all lie generally along a common line.

At step 204, the processor 116 generates an image from the first ultrasound data. According to an embodiment, the image may include a volume-rendered image. The term “volume-rendered image” is defined to include a two-dimensional representation of three-dimensional data. Typically, each sample point or voxel within the volume is assigned an opacity or weight. Then, through a technique such as ray-casting, pixels are assigned a value based on a combination of voxels values along rays originating from a focal point. Other embodiments may use different techniques to generate volume-rendered images.

According to other embodiments, at step 204, other types of images may be generated from the volumetric data. For example, according to an embodiment the image may include a slice of volumetric ultrasound data. Those skilled in the art will appreciate that this type of image closely resembles an image generated from two-dimensional ultrasound data. According to an embodiment, a user may be able to select the slice or plane of the image that is generated at step 204. For example, a user may be able to adjust the position of a cut-plane through the volume in order to determine the anatomical structures or features included in the image. At step 206, the image is displayed on the display device 118.

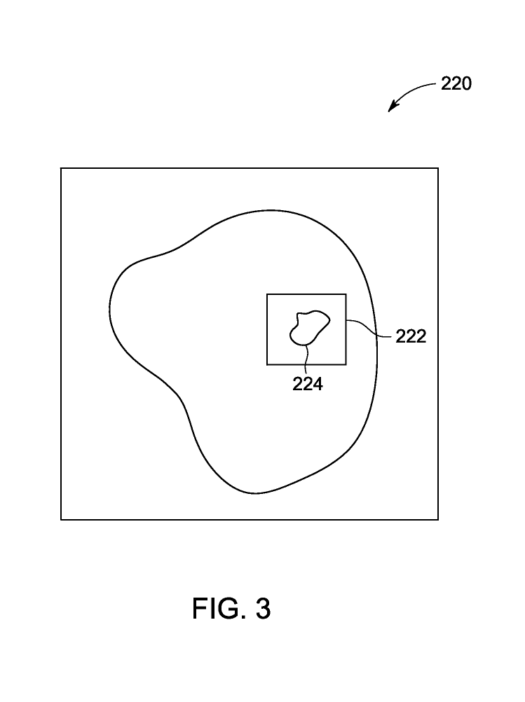

FIG. 3 is a schematic representation of a first image 220. The first image 220 may be an image or frame of a live image. A region-of-interest (ROI) 222 is shown surrounding a structure 224 in the first image 220. FIG. 3 will be described in more detail with respect to the method 200.

If additional first ultrasound data is required at step 207, the method 200 returns to step 202, where additional first ultrasound data is acquired. The method 200 may iteratively cycle through steps 202, 204, 206, and 207 multiple times. According to an embodiment, the most recently acquired image may replace the image that was displayed during the previous iteration of steps 202, 204, 206, and 207. By cycling through steps 202, 204, 206, and 207 multiple times, the method 200 may result in the display of a first sequence of images. Collectively, the displaying of the first sequence of images in this manner is often referred to as displaying a live or real-time image.

Referring now to FIGS. 1, 2, and 3, at step 208, an acquisition target is selected from the first sequence of images. The user may input commands through the user interface 115 in order to select a region-of-interest (ROI) such as the ROI 222. For example, the user may control both the size and the position of the region-of-interest 222. According to an exemplary embodiment, the acquisition target may include the region-of-interest 222. According to an embodiment, the first ultrasound data may include data of a relatively large volume in comparison with the size of the acquisition target. By acquiring a relatively large volume of ultrasound data, it is easy for the user to orient himself within the first image 220 and confidently identify the intended acquisition target. For example, the first image 220 may be used as an overview image in order to help the user select the desired acquisition target.

The acquisition target may be selected in different ways at step 208 according to other embodiments. For example, the user may identify just a structure, such as the structure 224. According to an embodiment, the processor 116 (shown in FIG. 1) may automatically segment the user-identified structure and place an appropriate ROI around the structure 224. According to other embodiments, the processor 116 may implement an object recognition algorithm in order to identify the structure within the first image. The structure may include any anatomical structure of interest, but according to an exemplary embodiment, the structure may be a portion of the patient's heart.

At step 210, the processor 116 automatically configures one or more acquisition parameters based on the acquisition target selected during step 208. The acquisition parameters are the settings that control the ultrasound data that will be acquired by the probe 105. The acquisition parameters control the ultrasound beams, which in turn control which portions of a patient's anatomy are imaged. For example, the acquisition parameters may control the position of the plane that is acquired when acquiring two-dimensional ultrasound data and the acquisition parameters may control the position and size of the volume that is acquired when acquiring volumetric ultrasound data. Non-limiting examples of acquisition parameters include: beam depth, beam steering angles, beam width, and beam spacing. The processor 116 configures the acquisition parameters in order to enable the acquisition of additional ultrasound data including the acquisition target that was identified during step 208.

At step 212, the processor 116 implements the acquisition parameters that were configured during step 210 in order to acquire second ultrasound data. According to an embodiment, the second ultrasound data may be of a smaller volume than the first ultrasound data. By acquiring a smaller volume of data, it is possible for the processor 116 to acquire data with higher temporal resolution and potentially higher spatial resolution as well. Higher temporal resolution data enables the user to view a live or dynamic image with a higher frame rate. Higher spatial resolution ultrasound data allows for the generation of higher resolution images. For example, images with higher spatial resolution allow the user to discern smaller details within the acquisition target. According to another embodiment, the second ultrasound data may include two-dimensional ultrasound data.

At step 214, the processor 116 generates an image from the second ultrasound data. Then, at step 216, the processor 116 displays the image generated from the second ultrasound data on the display device 118. If additional second ultrasound data is desired at step 217, then the method returns to step 212. In a manner similar to that previously described with respect to steps 202, 204, 206, and 207, the method 200 may iteratively repeat steps 212, 214, 216, and 217 multiple times in order to generate and display a second sequence of images. Collectively, the second sequence of images forms a second live ultrasound image. According to an embodiment, the acquisition parameter configured during step 210 was selected in part to give the second sequence of images a higher frame-rate than the first sequence of images. According to other embodiments, the individual images in the second sequence of images may also have higher spatial resolution than the individual images in the first sequence of images. If no additional second ultrasound data is desired at step 217, then the method 200 ends.

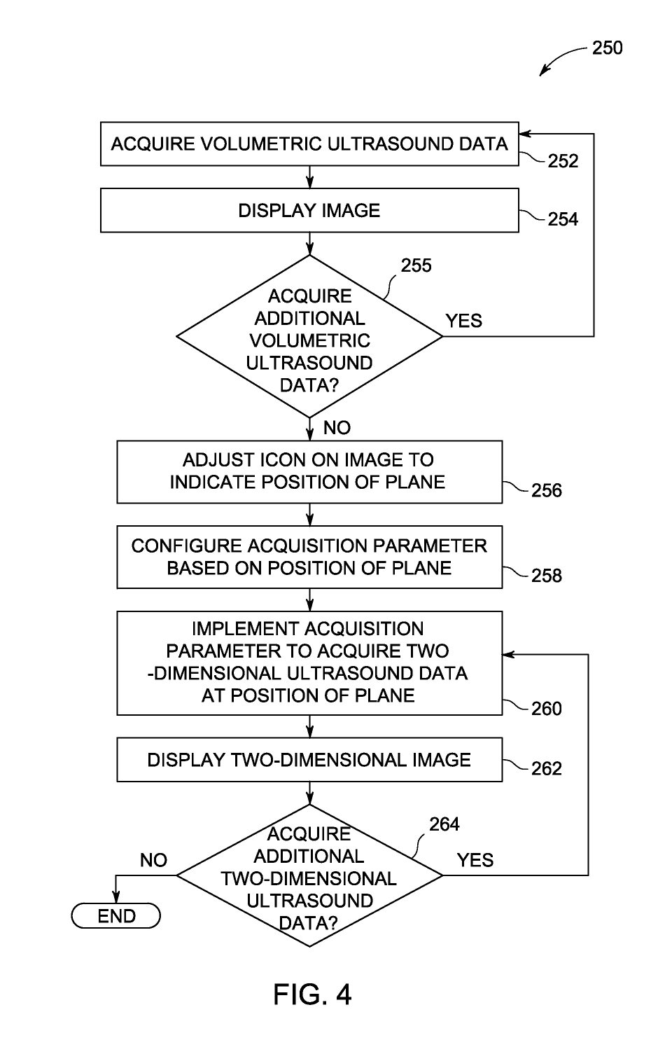

FIG. 4 is a flow chart illustrating a method 250 in accordance with an embodiment. The individual blocks represent steps that may be performed in accordance with the method 250. The technical effect of the method 250 is the display of a two-dimensional image selected by adjusting an icon on an image generated from volumetric ultrasound data. The steps of the method 250 will be described according to an exemplary embodiment where the steps are performed with the ultrasound imaging system 100 (shown in FIG. 1),

Referring to FIGS. 1 and 4, at step 252, the processor 116 acquires volumetric ultrasound data. Next, at step 254, the processor 116 displays an image generated from the volumetric ultrasound data. If additional volumetric ultrasound data is required at step 255, the method returns to step 252. According to an embodiment, the method 250 may iteratively cycle through steps 252, 254, and 255 multiple times. Each time the method cycles through steps 252, 254, and 255, a new image is generated based on the most recently acquired volumetric ultrasound data. Collectively, the sequence of images displayed at step 254 through multiple iterations form a live or dynamic ultrasound image. If no additional volumetric ultrasound data is required, then the method advances to step 256.

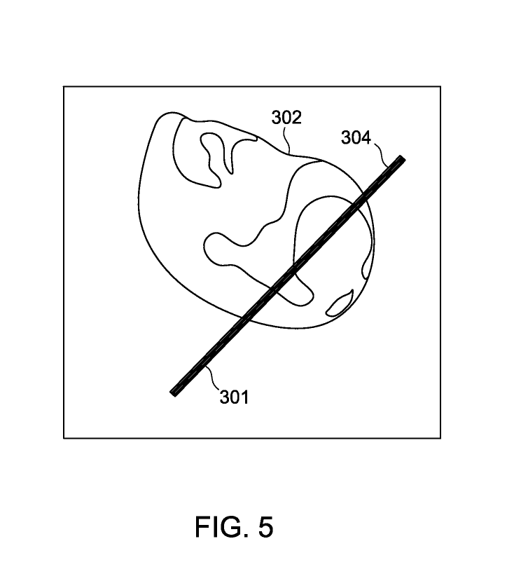

FIG. 5 is a schematic representation of a volume-rendered image and an icon in accordance with an embodiment. The icon 301 is represented as an overlay on top of the volume-rendered image 302. The icon 301 includes a line 304 in accordance with an embodiment.

Referring now to FIGS. 4, 5, and 1, at step 256, the user adjusts the position of the icon 301. The line 304 represents the position of a plane. According to an exemplary embodiment, the first plane intersects the volume-rendered image 302 along the line 304. The processor 116 may limit the permissible locations of the icon 301 on the display device 118 to only the positions where the plane corresponding to the icon's location would intersect the probe 105.

According to an embodiment, a two-dimensional rendering of the plane may be displayed based on the volumetric data. The use of the two-dimensional rendering of the plane will be described hereinafter.

At step 258, the processor 116 configures an acquisition parameter based on the position of the plane as determined by the position of the icon 301 on the volume-rendered image 302. The acquisition parameters are configured in order to enable the acquisition of two-dimensional data including the first plane. The acquisition parameters determine the location from which the two-dimensional ultrasound data is acquired. The acquisition parameters are selected to enable the acquisition of two-dimensional ultrasound data from the desired plane within a subject. Examples of acquisition parameters include: beam depth, beam steering angle, beam width, beam spacing, and the like.

At step 260, the processor 116 implements the acquisition parameters configured during step 258. As discussed hereinabove, the acquisition parameters may have been selected to enable the ultrasound imaging system 100 to acquire two-dimensional ultrasound data of a plane selected by positioning the icon 301. At step 262, the processor 116 displays a two-dimensional image on the display device 118. According to other embodiments, the acquisition parameters may be configured to enable the ultrasound imaging system to acquire ultrasound data for two or more planes. For example, other embodiments may have an icon with multiple lines, where each line represents a plane.

Referring to FIG. 4, the method 250 may iteratively repeat steps 260 and 262 in order to acquire and display a live two-dimensional image. For example, if additional two-dimensional ultrasound data is required at step 264, then the method 250 returns to step 260. For example, each of the two-dimensional images may be used as an image frame within a live or dynamic two-dimensional image.

As described hereinabove, in an embodiment a slice based on the volumetric ultrasound data may be displayed at the same time as a two-dimensional image based on two-dimensional ultrasound data. The user may compare the slice based on the volumetric ultrasound data to the two-dimensional image in order to confirm that the two-dimensional image contains the intended anatomical structure. According to an embodiment, the two-dimensional image may have better spatial resolution than the image generated from the volumetric ultrasound data, thus making the two-dimensional image more diagnostically useful. According to embodiments where a live volume-rendered image and a live two-dimensional image are displayed, the live two-dimensional image may exhibit higher temporal resolution than the live volume-rendered image. The higher spatial resolution allows the user to identify motion of the structure more accurately.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of ultrasound imaging comprising:
displaying a first sequence of images generated from first ultrasound data, wherein the first ultrasound data comprises volumetric ultrasound data;
selecting an acquisition target from the first sequence of images;
automatically configuring an acquisition parameter based on the selected acquisition target;
implementing the acquisition parameter to acquire second ultrasound data of the acquisition target; and
displaying a second sequence of images generated from the second ultrasound data, wherein the second sequence of images is of a higher frame rate than the first sequence of images.

2. The method of claim 1, wherein the second sequence of ages has a higher spatial resolution than the first sequence of images.

3. The method of ultrasound imaging of claim 1, wherein the acquisition target comprises a region-of-interest.

4. The method of ultrasound imaging of claim 1, wherein the acquisition parameter comprises one or more of beam depth, beam width, and beam spacing.

5. The method of ultrasound imaging of claim 1, wherein the acquisition parameter comprises a mode.

6. The method of claim 1, wherein the second ultrasound data comprises two-dimensional ultrasound data.

7. The method of ultrasound imaging of claim 1, wherein the second ultrasound data comprises second volumetric ultrasound data of a smaller volume than the first ultrasound data.

8. The method of ultrasound imaging of claim 1, further comprising acquiring reference ultrasound data, determining a relative displacement between the first ultrasound data and the reference ultrasound data, and automatically adjusting the acquisition parameter to compensate for the relative displacement before said implementing the acquisition parameter to acquire the second ultrasound data.

9. The method of claim 1, wherein said displaying the second sequence of images comprises displaying each of the second sequence of images in real-time while the second ultrasound data is being acquired.

10. A method of ultrasound imaging comprising:
acquiring volumetric ultrasound data;
displaying an image generated from the volumetric ultrasound data;
adjusting the position of an icon on the image to control a position of a plane;
automatically configuring an acquisition parameter based on the position of the plane;
implementing the acquisition parameter to acquire two-dimensional ultrasound data at the position of the plane; and
displaying a two-dimensional image generated from the two-dimensional ultrasound data.

11. The method of ultrasound imaging of claim 10, wherein the icon comprises a line indicating the intersection of the plane and the first image.

12. The method of ultrasound imaging of claim 10, further comprising displaying an image of the plane generated from the volumetric ultrasound data while said implementing the acquisition parameter to acquire the two-dimensional ultrasound data at the position of the plane.

13. The method of ultrasound imaging of claim 10, further comprising limiting with the processor the possible positions of the icon on the first image so that the icon may only be positioned where a plane corresponding to the icon would intersect a location of a probe.

14. The method of ultrasound imaging of claim 10, further comprising automatically acquiring second two-dimensional ultrasound data, said second two-dimensional ultrasound data comprising data of a second plane that is distinct from the plane.

15. The method of ultrasound imaging of claim 14, further comprising displaying a second two-dimensional image generated from the second two-dimensional ultrasound data at generally the same time as said displaying the two-dimensional image.

16. An ultrasound imaging system comprising:
a probe adapted to scan a volume of interest;
a display device; and
a processor in electronic communication with the probe and the display device, wherein the processor is configured to:
control the probe to acquire first ultrasound data, said first ultrasound data comprising volumetric ultrasound data;
display a first image based on the first ultrasound data on the display device;
automatically configure an acquisition parameter based on the selection of an acquisition target in the first image;
implement the acquisition parameter to acquire second ultrasound data of the acquisition target, where the second ultrasound data is of higher temporal resolution than the first ultrasound data; and
display an image generated from the second ultrasound data on the display device.

17. The ultrasound imaging system of claim 16, wherein the processor is further configured to implement an object-recognition algorithm in order to assist in the selection of the acquisition target.

18. The ultrasound imaging system of claim 17, wherein the processor is further configured to use the results of implementing the object-recognition algorithm in order to automatically configure the acquisition parameter.

19. The ultrasound imaging system of claim 16, wherein the processor is configured to automatically configure the acquisition parameter selected from the group consisting of beam depth, beam width, beam direction, and beam spacing.

20. The ultrasound imaging system of claim 16, wherein the processor is configured to display a sequence of images generated from the second ultrasound data.
